(19) United States
(12) Patent Application Publication
(10) Pub. No.: US 2026/0198975 A1
(43) Pub. Date: Jul. 16, 2026

(54) Arthroplasty Stem Attachment to Expandable Intramedullary Nail

(71) Applicant: Ossura Medical, Norristown, PA (US)

(72) Inventors: Zachary Wallace, Philadelphia, PA (US); Mark Hodgeland, Norristown, PA (US); Lauren McGinley, Phoenixville, PA (US)

(73) Assignee: Ossura Medical, Norristown, PA (US)

(21) Appl. No.: 19/432,124

(22) Filed: Dec. 24, 2025

Related U.S. Application Data
(60) Provisional application No. 63/738,525, filed on Dec. 24, 2024.

(51) Int. Cl. A61B 17/72

(52) U.S. Cl. CPC A61B 17/72

(57) ABSTRACT

An orthopedic device is described that serves as an attachment to an intramedullary nail to allow for acceptance of another implant. The attachment device includes a male end or a corresponding female end that mates with the intramedullary nail through a threaded mechanism or other means and a female end or the corresponding male end for acceptance of any type of stemmed implant such as another IM nail, hip arthroplasty stem, or knee arthroplasty stem, among others. The attachment body has multiple sizes to accommodate both the increase in diameter of the expandable intramedullary nail as well as the differences in shape and size of the other implant.

### CROSS-REFERENCE TO RELATED APPLICATIONS

This application is a U.S. Non-Provisional Utility Patent Application entitled, “Arthroplasty Stem Attachment to Expandable Intramedullary Nail” which claims priority to U.S. Provisional Patent Application No. 63/738,525 , filed on Dec. 24, 2024 entitled, “Arthroplasty Stem Attachment to Expandable Intramedullary Nail” the contents of which are hereby fully incorporated by reference.

### FIELD OF THE INVENTION

The present invention relates to an orthopedic device that is an attachment to an expandable intramedullary nail to allow for acceptance of another implant. The attachment body includes a male end that mates only with the expandable intramedullary nail through a threaded mechanism or other means and another female end for acceptance of any type of stemmed implant such as another IM nail, hip arthroplasty stem, or knee arthroplasty stem, among others. The attachment body has multiple sizes to accommodate both the increase in diameter of the expandable intramedullary nail as well as the differences in shape and size of the other implant.

### BACKGROUND OF THE INVENTION

A mummy from ancient Egypt was found to have an insert in its left knee that dates to 3500 years ago. It is believed that this insert was inserted after the man had died and was used to stabilize the leg during movement prior to his burial. This earliest medical device was more primitive than inserts that are found today.

An intramedullary nail (IM nail) or inter-locking nail or Küntscher nail (without proximal or distal fixation), is a metal rod forced into the medullary cavity of a bone. Intramedullary nails may be implanted in the bones of humans. A purpose of such nails may be to increase the longitudinal strength of the implanted bone allowing a patient to stand more rapidly as some of the longitudinal force on the nail is born by the nail (and not just the bone). IM nails have long been used to treat fractures of long bones of the body, such as the femur or humerus (or the radius, ulna, tibia, or fibula). Gerhard Küntscher is credited with the first use of this device in 1939, during World War II, for soldiers with fractures of the femur. Prior to that, treatment of such fractures was limited to traction or plaster, both of which required long periods of inactivity. IM nails resulted in earlier return to activity for the soldiers, sometimes even within a span of a few weeks, since they share the load with the bone, rather than entirely supporting the bone.

Stainless steel was used on the earliest IM nails, but with the development of new stronger polymers and other biocompatible plastics and better and stronger metals, lighter and stronger inserts have been developed and used more frequently.

If the intramedullary nail is implanted in a human who is not fully grown, the growth of the human will normally increase the desired length of the intramedullary nail. For example, if the intramedullary nail is implanted in the leg of a human child, the desired length of the nail will increase as the child grows and its leg becomes longer. However, the length of the nail does not automatically increase as the human grows leading to a mismatched actual length and a desired length.

The increase in the length of the leg of the child will result in the intramedullary nail then implanted in the leg becoming too short for the leg having the increased length. If the intramedullary nail is not replaced and lengthened, then the nail will most likely interfere with the proper longitudinal growth of the leg leading to a leg that is shorter in length. Replacement surgery to change out a shorter IM nail for a longer one is undesirable for a number of reasons, including multiple surgeries, which leads to additional scar tissue, an increased possibility of infection, damage to tissue, damage to bone, damage to joints, subjecting individuals to multiple rounds of anesthesia, and other potential drawbacks from having to perform additional surgeries.

Additionally, in periprothetic fractures (fractures around hip or knee implant), it would be desirable to adjust nails to the appropriate length allowing attachment to and interface with the hip/knee stem to create one whole construct. The currently used methods use multiple means of fixation (plates, nails with plates, etc) that are simply inadequate. Additionally, it would be desirable to be able to use the nails in standard fractures where the surgeon wants controlled compression. It is with all of these limitations/desires in mind that the present invention was developed.

### BRIEF SUMMARY OF THE INVENTION

The present invention relates to an attachment device that is attached to an expandible-extendible intramedullary nail or to any intermedullary nail, and methods for using the attachment device attached to the intramedullary expandible-extendible nails, wherein the attachment device allows for the attachment of other devices to the intramedullary nails to create a single multi-part construct.

In an embodiment, the attachment device is an elongate tubular device that attaches to an intramedullary nail, which includes an elongate tubular base section. An elongate tubular assembly component is connected to the base section. The assembly component has an inner assembly surface and helical assembly threads formed on the inner assembly surface. The assembly component and helical assembly threads each have a longitudinal central axis. The longitudinal central axes of the assembly component and helical assembly threads coincide with one another.

The elongate tubular distal rod has a means of allowing acceptance of another implant. In an embodiment, the attachment body includes a male end or a corresponding female end that mates only with the expandable intramedullary nail (e.g., to the elongate tubular distal rod) through a threaded mechanism or other means and another female end (or the corresponding male end) for acceptance of any type of stemmed implant such as another IM nail, hip arthroplasty stem, or knee arthroplasty stem, among others. The attachment body has multiple sizes to accommodate both the increase in diameter of the expandable intramedullary nail as well as the differences in shape and size of the other implant.

### BRIEF DESCRIPTION OF THE SEVERAL VIEWS OF THE DRAWING

FIG. 1A shows a front sectional view of the cup designed attachment device.

FIG. 1B shows a top down perspective view of the cup designed attachment device.

FIG. 1C shows a front view of the cup designed attachment device.

FIG. 2A shows a front sectional view of the lipstick configuration attachment device.

FIG. 2B shows a side perspective view of the lipstick configuration attachment device.

FIG. 2C shows a front perspective view of the lipstick configuration attachment device.

FIG. 3A shows a front sectional view of the modified lipstick configuration attachment device.

FIG. 3B shows a side perspective view of the modified lipstick configuration attachment device.

FIG. 3C shows a front perspective view of the modified lipstick configuration attachment device.

### DETAILED DESCRIPTION OF THE INVENTION

In an embodiment, the present invention relates to an orthopedic device that is an attachment to an intramedullary nail. This orthopedic device which attaches to the intramedullary nail allows for acceptance of another implant. The attachment body includes a male end that mates only with the expandable intramedullary nail through a threaded mechanism or other means and another female end for acceptance of any type of stemmed implant such as another IM nail, hip arthroplasty stem, or knee arthroplasty stem, among others. The attachment body has multiple sizes to accommodate both the increase in diameter of the expandable intramedullary nail as well as the differences in shape and size of the other implant.

In an embodiment, the present invention relates to a female feature at the base of the design so it can accept another nail or implant. In a variation, the base of the design wouldn't need any key feature or threads or anything, but just a simple cavity at the end of the nail. The weight of the patient pushing down and the screws keeping the implant in place would then create a “single construct”. In a variation, it is believed that this design would be incredibly useful in periprosthetic fractures.

It should be understood that when a female or male feature is discussed as being on either the expandable intramedullary nail or on the orthopedic device (attachment device) to allow each to be operationally and correspondingly connected to each other, other embodiments are also contemplated wherein the female or male feature is on the other device. As long as they allow each to correspondingly connect with each other, that embodiment is contemplated as a potential embodiment of the present invention.

In an embodiment, the present invention relates to other possible ways of attaching the attachment to the nail. For example, the connection means includes but is not limited to the use of threaded, press fit, snap fit or other mechanisms. It should also be understood that when a particular means of attaching the attachment to the intermedullary nail is described that the corresponding feature that allows the connection to be made can occur on the other device. For example if a screw type mechanism is used and the male end is on the intermedullary nail and the female end is on the attachment, it is contemplated and within the scope of the invention that the female end may be on the intermedullary nail and the male end may be on the attachment.

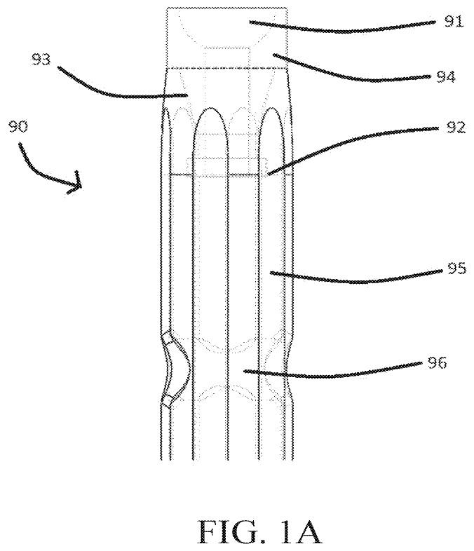

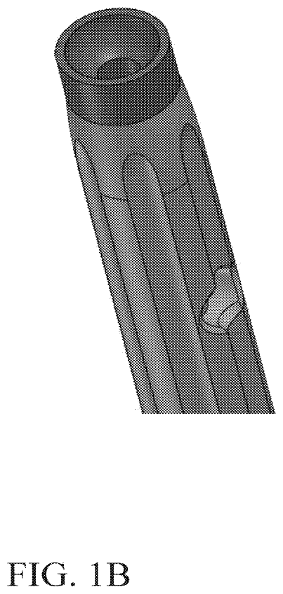

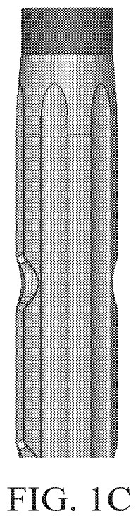

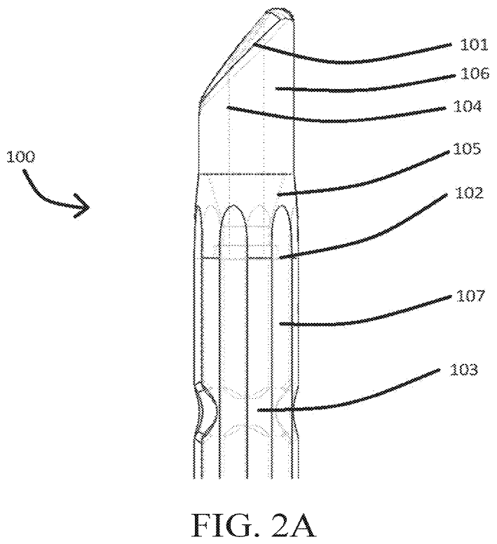

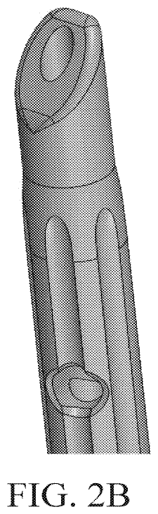

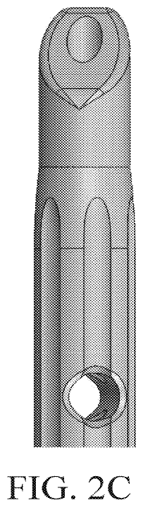

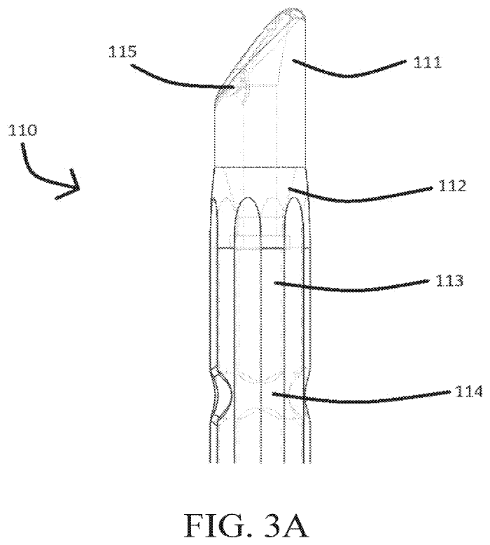

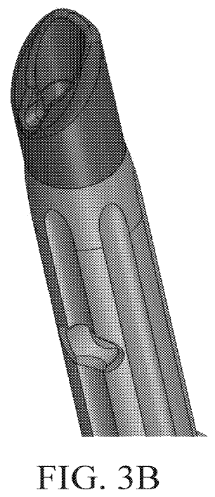

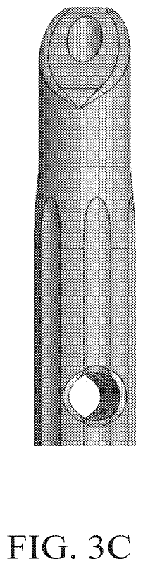

Three different embodiments are shown in the FIGS. 1A-1C, 2A-2C, and 3A-3C with each of FIGS. 1, 2 and 3 representing different embodiments. FIG. 1A represents a cup design version of one of the embodiments, whereas FIG. 2A and FIG. 3A represent a lipstick configuration and a modified lipstick configuration, respectively of the various attachments.

FIG. 1A shows the combination 90 of the attachment device 94 and the expandable intramedullary nail 95. A male end or tapered outer portion 93 at the distal portion of the attachment device 94 allows the attachment device 94 to be positioned inside expandable intramedullary nail 95, wherein expandable intramedullary nail 95 has a female end or tapered inner portion designed and configured to ideally accommodate the male end or tapered outer portion 93 of the attachment device. The distal end 92 of the attachment device 94 is shown in FIG. 1A. It should be understood that any of a plurality of means can be used to attach the attachment device 94 with the expandable intramedullary nail 95 including but not limited to a pressured fit, a snap fit, biocompatible glue and a screw mechanism. The pressured fit (which is essentially just the attachment device abutting an inner portion of the expandable intramedullary nail 95 until the distal end 92) and the snap fit may allow for free rotation of the attachment device 94 about an axis that is the longitudinal direction of the attachment device 94 and the expandable intramedullary nail 95. The pressured fit generally will rely on the weight of a patient to keep the attachment device affixed to the expandable intramedullary nail 95. Moreover, because the expandable intramedullary nail 95 is likely to be screwed into a bone (for example, a femur) at screw hole 96 (or at any of a plurality of other locations), the expandable intramedullary nail 95 will be affixed to the bone (e.g., the femur) at a distal location of 95, thereby meaning that the attachment device 94 does not necessarily need to be the expandable intramedullary nail permanently affixed to the expandable intramedullary nail 95. This is because at the proximal portion of the attachment device 94, there is a socket (cup) 91 (or a proximal female end) which is ideally adapted and configured to accommodate another medical device (e.g., a hip stem, a shoulder stem, or knee arthroplasty stem), which applies pressure from the other direction. Thus, the expandable intramedullary nail 95 compresses the attachment device from one direction and the hip compresses the attachment device 94 from the other.

Thus, the attachment device 94 of the present invention and as shown in FIG. 1A has some or all of the following features:
a) Allows mating with distal portion of a hip stem or other medical device such as a knee joint, an elbow joint, an ankle prosthesis, or other similar joint prostheses.
b) May be made from metal such as stainless steel, a titanium containing alloy, a cobalt containing alloy, or a semi-rigid plastic biocompatible polymer.
c) Designed to allow free rotation within the expandable intramedullary nail 95; or
d) Alternatively the method by which the attachment device is affixed to the expandable intramedullary nail 95 may be by threading (screwed together) or attached by a biocompatible glue, in which case, the attachment device 94 will not freely rotate.

FIGS. 1B and 1C show a perspective view and a side view, respectively of the attachment device shown in FIG. 1A. In FIG. 1B, the socket (cup) 91 (or proximal female end) is especially prominent. It should be noted that as shown, the socket (cup) 91 (or proximate female end) has a hole at the bottom of the cup that allows passage via a passageway of a guide wire through the attachment device and optionally through the expandable intramedullary nail 95. The guide wire (not shown) may be present prior to the installation of the expandable intramedullary nail 95 and the attachment device 94, which allows each and both to be properly positioned in a patient that requires the combination 90 of the expandable intramedullary nail 95 and the attachment device 94.

FIG. 2A shows a lipstick configuration 100, which comprises a combination of the expandable intramedullary nail 107 and the attachment device 106. In this embodiment, the attachment device 106 has a lipstick shaped configuration that results at least partially from the shape of slanted edge 101. It should be understood that the slanted edge 101 may be designed to have any number of angles that allow the slanted edge to be ideally suited for its purpose. For example, if a hip stem is to be attached to the proximal end of the attachment device 106, the angle of the slanted edge 101 should be of an angle that allows the hip stem to be properly oriented so that the hip stem will fit at the correct angle (and length) into the socket of the hip/pelvis.

In FIG. 2A, there is a passageway 104 (similar to FIG. 1A) that allows the lipstick configuration 100 to be placed in the patient as needed in an operationally correct manner via a guide wire (not shown). That is, the guide wire allows placement of the expandable intramedullary nail 107 and the attachment device 106. The guide wire may be inserted after optional reaming of the bone so that the guide wire can be passed into the femur (or whatever bone is having the intramedullary nail inserted into the intra-medullary canal). Subsequently the guide wire can be used to guide the expandable intramedullary nail 107 and the attachment device 106 into the correct location. After the optional attachment of appropriately positioned screws onto the expandable intramedullary nail 107 (for example, at screw hole 103), the attachment device can then have a prothesis (e.g., a hip stem or knee arthroplasty stem) added to the attachment device. Depending on how the attachment device 106 is attached to the expandable intramedullary nail 107, the distal end 102 of the attachment device 106 and the attachment device 106 may rotate in either a clockwise or counterclockwise direction around the longitudinal axis of the expandable intramedullary nail 107 and the attachment device 106. If the attachment device 106 is affixed via screws or a biocompatible glue, the attachment device 106 will be precluded from rotating. However, should the attachment device 106 be affixed to the expandable intramedullary nail 107 via pressure or via a snap fit (depending on the type of snap fit), the attachment device 106 is likely to be freely flexible. The attachment device may have a male end or tapered outer surface/portion 105 that allows it to be operationally connected (the shapes are designed to accommodate each other) to a female end or tapered inner surface/portion of the expandable intramedullary nail 107.

FIGS. 2B and 2C show two separate perspective views of the lipstick configuration shown in FIG. 2A. It should be noted that there is a socket/cup (or a proximal female end) present at the proximal end of the attachment device 106 that allows the prothesis to be attached to the attachment device.

The features of attachment device 106 as shown in FIGS. 2A-2C comprise one or more of the following:
a) The attachment device 106 mates with a distal portion of a hip stem by wedging on one side.
b) The attachment device 106 may be made from metal or a semi-rigid plastic (as described above)
c) The attachment device 106 is designed in way that it can freely rotate within any nail or an expandable intramedullary nail.
d) Alternatively, if the method of attachment is by threading (or by glue), the attachment device 106 cannot freely rotate.

FIG. 3A is similar to FIG. 2A. However, FIG. 3A shows a modified lipstick configuration 110. The modified lipstick configuration 110 shows many of the same features that are shown in FIG. 2A, with the principal difference being a cored out center region 115 on the distal end of the attachment device 111, that allows for the attachment device 111 to be better wedged against a hip stem that is attached to the attachment device 111. The cored out center region 115 can be best seen in the perspective view of FIG. 3B. As is similar to FIG. 2A, FIG. 3A contains the expandable intramedullary nail 113, the male end or tapered outer surface 112 of attachment device 111 that allows the attachment device 111 to be operationally connected to the expandable intramedullary nail 113 which has the correspondingly featured female end or tapered inner surface that is designed to allow the attachment device 111 and the expandable intramedullary nail 113 to be connected in a manner that allows a snug fit. As was described with FIG. 2A, FIG. 3A allows the attachment device 111 and the expandable intramedullary nail 113 to be affixed via a biocompatible glue or via screws. Alternatively, a snap fit or a pressure fit may occur allowing the attachment device 111 and the expandable intramedullary nail 113 to abut each other. The expandable intramedullary nail 113 also comprises the screw hole 114 that allows attachment of the expandable intramedullary nail 113 to the bone via one or more screws.

FIG. 3C shows a front perspective view of the embodiment shown in FIGS. 3A and 3B. The features of attachment device 111 as shown in FIGS. 3A-3C comprise one or more of the following:
a) The attachment device 111 is similar to the lipstick configuration design of FIG. 2A but with a cored-out center to wedge better against a hip stem or some other prothesis.
b) The attachment device 111 may be made from metal or a semi-rigid plastic such as stainless steel, a titanium based alloy, a cobalt based alloy, or a biocompatible polymer semi-rigid plastic.
c) The attachment device 111 as shown can freely rotate within a intramedullary nail or an expandable intramedullary nail.
d) Alternatively, the attachment device 111 uses an attachment method that is threaded. If this design is used threaded, the attachment device 111 cannot freely rotate.

The expandible-extendible intramedullary nail has the features that are described in U.S. Provisional Patent Application No. 63/553,150 filed Feb. 14, 2024, which is herein incorporated by reference in its entirety. These features include:

The expandible-extendible intramedullary nail includes an elongate tubular base section. An elongate tubular assembly component is connected to the base section. The assembly component has an inner assembly surface and helical assembly threads formed on the inner assembly surface. The assembly component and helical assembly threads each have a longitudinal central axis. The longitudinal central axes of the assembly component and helical assembly threads coincide with one another.

An elongate tubular distal rod is connected to the assembly component. The distal rod has a longitudinal central axis that coincides with the longitudinal central axis of the assembly component.

An elongate tubular driving nut is connected to the distal rod and assembly component. The driving nut has an outer nut surface and helical nut threads formed on the outer nut surface. The helical nut threads and distal rod each have a longitudinal central axis. The longitudinal central axes of the helical nut threads and distal rod coincide with one another. The helical nut threads correspond to the helical assembly threads.

The driving nut is rotatable about the longitudinal central axis of the helical nut threads such that the rotation causes helical displacement of the helical nut threads relative to the helical assembly threads. This, in turn, causes longitudinal displacement of the distal rod relative to the assembly component.

The expandible-extendible intramedullary nail may be lengthened or shortened while in the body of the human. Thus, replacing the intramedullary nail is not necessary if the part of the human, such as a leg, in which the intramedullary nail is implanted changes its longitudinal dimension. In the event of such a change, the longitudinal dimension of the intramedullary nail may be changed in vivo to accommodate the change in the longitudinal dimension of the part of the human in which the intramedullary nail is implanted.

The expandible-extendible intramedullary nail has mechanisms which cause the change in the longitudinal dimension of the nail. These mechanisms are contained within the outer surface of the intramedullary nail thereby giving the nail a smooth, even outer contour. Consequently, movement of these mechanisms does not cause associated forces on the tissue that adjoins the outer surface thereby reducing or preventing entirely irritation of this tissue.

In an embodiment, the present invention relates to an attachment device configured and designed to be operationally connected to an intramedullary nail on a first side of the attachment device, and operationally connected to an implant or prothesis on an opposite second side of the attachment device.

In a variation, the first side of the attachment device comprises a male end. In a variation, the first side of the attachment device comprises a female end. In a variation, the intramedullary nail is an expandable intramedullary nail. In a variation, the male end comprises at least a tapered portion. In a variation, the female end comprises at least a tapered portion.

In a variation, the implant or prothesis comprises a hip stem or a knee arthroplasty stem. In a variation, the implant or prothesis comprises a hip stem or a knee arthroplasty stem. In a variation, the attachment device is connected to the intramedullary nail via one or more methods selected from the group consisting of pressure, glue, screws, and snap-fitting. In a variation, the attachment device is connected to the intramedullary nail via one or more methods selected from the group consisting of pressure, glue, screws, and snap-fitting. In a variation, the attachment device is in the shape of a cup configuration, a lipstick configuration, or a modified lipstick configuration. In a variation, the attachment device is made of a metal alloy or a semi rigid plastic polymer. In a variation, the metal alloy is stainless steel, a titanium alloy, or a cobalt alloy.

In a variation, the attachment device is configured and designed to freely rotate inside the intramedullary nail. In a variation, the attachment device is configured and designed to freely rotate inside the expandable intramedullary nail.

In an embodiment, the present invention relates to a method of introducing an attachment device into a patient, the attachment device configured and designed to be operationally connected to an expandable intramedullary nail on a first side of the attachment device, and operationally connected to an implant or prothesis on an opposite second side of the attachment device; said method comprising:
a) Creating an incision on a patient in need of the expandable intramedullary nail;
b) Exposing a bone on which the expandable intramedullary nail is to be inserted;
c) Drilling into a medullary canal of the bone to allow passage via a passageway of the expandable intramedullary nail to the medullary canal;
d) Inserting a guide wire into the medullary canal via the passageway;
e) Using the guide wire to insert the expandable intramedullary nail into the medullary canal of the bone;
f) Optionally screwing the expandable intramedullary nail to the bone;
g) Attaching the attachment device to the expandable intramedullary nail using the first side of the attachment device; and
h) Removing the guide wire.

In a variation of the method, the method further comprises inserting the implant or prothesis on the opposite second side of the attachment device. In a variation, the implant or prothesis comprises a hip stem or a knee arthroplasty stem. In a variation, attaching the attachment device to the expandable intramedullary nail occurs via one or more methods selected from the group consisting of pressure, glue, screws, and snap-fitting. In a variation, the method is used for a patient that has a periprosthetic fracture.

In an embodiment, the present invention relates to an apparatus that comprises an attachment device and an expandable intramedullary nail, the attachment device being operationally connected to the expandable intramedullary nail via one or more methods selected from the group consisting of pressure, glue, screws, and snap-fitting, the attachment device being made of a metal alloy or a semi rigid plastic polymer, wherein the metal alloy is stainless steel, a titanium alloy, or a cobalt alloy.

In a variation, the apparatus further comprises an implant or prothesis, the implant or prothesis comprising a hip stem or a knee arthroplasty stem.

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1. An attachment device configured and designed to be operationally connected to an intramedullary nail on a first side of the attachment device, and operationally connected to an implant or prothesis on an opposite second side of the attachment device.

2. The attachment device of claim 1, wherein the first side of the attachment device comprises a male end.

3. The attachment device of claim 1, wherein the intramedullary nail is an expandable intramedullary nail.

4. The attachment device of claim 2, wherein the male end comprises at least a tapered portion.

5. The attachment device of claim 1, wherein the implant or prothesis comprises a hip stem or a knee arthroplasty stem.

6. The attachment device of claim 3, wherein the implant or prothesis comprises a hip stem or a knee arthroplasty stem.

7. The attachment device of claim 1, wherein the attachment device is connected to the intramedullary nail via one or more methods selected from the group consisting of pressure, glue, screws, and snap-fitting.

8. The attachment device of claim 3, wherein the attachment device is connected to the intramedullary nail via one or more methods selected from the group consisting of pressure, glue, screws, and snap-fitting.

9. The attachment device of claim 1, wherein the attachment device is in the shape of a cup configuration, a lipstick configuration, or a modified lipstick configuration.

10. The attachment device of claim 1, wherein the attachment device is made of a metal alloy or a semi rigid plastic polymer.

11. The attachment device of claim 10, wherein the metal alloy is stainless steel, a titanium alloy, or a cobalt alloy.

12. The attachment device of claim 1, wherein the attachment device is configured and designed to freely rotate inside the intramedullary nail.

13. The attachment device of claim 3, wherein the attachment device is configured and designed to freely rotate inside the expandable intramedullary nail.

14. A method of introducing an attachment device into a patient, the attachment device configured and designed to be operationally connected to an expandable intramedullary nail on a first side of the attachment device, and operationally connected to an implant or prothesis on an opposite second side of the attachment device; said method comprising:
a) Creating an incision on a patient in need of the expandable intramedullary nail;
b) Exposing a bone on which the expandable intramedullary nail is to be inserted;
c) Drilling into a medullary canal of the bone to allow passage via a passageway of the expandable intramedullary nail to the medullary canal;
d) Inserting a guide wire into the medullary canal via the passageway;
e) Using the guide wire to insert the expandable intramedullary nail into the medullary canal of the bone;
f) Optionally screwing the expandable intramedullary nail to the bone;
g) Attaching the attachment device to the expandable intramedullary nail using the first side of the attachment device; and
h) Removing the guide wire.

15. The method of claim 12, further comprising inserting the implant or prothesis on the opposite second side of the attachment device.

16. The method of claim 15, wherein the implant or prothesis comprises a hip stem or a knee arthroplasty stem.

17. The method of claim 15, wherein the attaching the attachment device to the expandable intramedullary nail occurs via one or more methods selected from the group consisting of pressure, glue, screws, and snap-fitting.

18. The method of claim 14, wherein the method is used for a patient that has a periprosthetic fracture.

19. An apparatus that comprises an attachment device and an expandable intramedullary nail, the attachment device being operationally connected to the expandable intramedullary nail via one or more methods selected from the group consisting of pressure, glue, screws, and snap-fitting, the attachment device being made of a metal alloy or a semi rigid plastic polymer, wherein the metal alloy is stainless steel, a titanium alloy, or a cobalt alloy.

20. The apparatus of claim 19, wherein the apparatus further comprises an implant or prothesis, the implant or prothesis comprising a hip stem or a knee arthroplasty stem.
